(19) United States
(12) Patent Application Publication
(10) Pub. No.: US 2019/0088019 A1
(43) Pub. Date: Mar. 21, 2019

(54) CALCULATION DEVICE FOR SUPERIMPOSING A LAPAROSCOPIC IMAGE AND AN ULTRASOUND IMAGE

(71) Applicant: KONINKLIJKE PHILIPS N.V., EINDHOVEN (NL)

(72) Inventors: Sven PREVRHAL, HAMBURG (DE); Jörg SABCZYNSKI, HAMBURG (DE)

(73) Assignee: KONINKLIJKE PHILIPS N.V., Eindhoven (NL)

(21) Appl. No.: 16/084,638

(22) PCT Filed: Mar. 15, 2017
(86) PCT No.: PCT/EP2017/056045

§ 371 Date: Sep. 13, 2018

(30) Foreign Application Priority Data
Mar. 16, 2016 (EP) 16160609.0

(51) Int. Cl. G06T 19/00; A61B 8/08; G06T 7/50; G06T 7/33; G06T 7/70; G06T 7/60; A61B 1/313; A61B 8/00; A61B 8/12; G06F 3/01; G06T 3/00; A61B 5/06

(52) U.S. Cl. CPC G06T 19/006; A61B 2090/365; G06T 7/50; G06T 7/337; G06T 7/70; G06T 7/60; A61B 1/3132; A61B 8/463; A61B 8/12; G06F 3/013; G06T 3/0093; A61B 5/066; A61B 8/0841; G06T 2207/10028; G06T 2207/10068; G06T 2207/10132; G06T 2207/20221; G06T 2207/30004; A61B 8/5261

(57) ABSTRACT

The present invention relates a calculation device for superimposing a laparoscopic image and an ultrasound image. The calculation device is configured for receiving a laparoscopic image, an ultrasound image and a depth image of a depth-sensing device. The calculation device extracts depth cue information from the depth image and uses the extracted depth cue information for superimposing the laparoscopic image and the ultrasound image thereby generating a superimposed image. The calculation device may use the spatial position and orientation of both the laparoscope and the ultrasound device to spatially co-register the devices relative to each other. This can then be used to present a correctly superimposed view rendering of both laparoscope and ultrasound image data. This merged view greatly facilitates the user in locating and positioning the ultrasound probe and the location of interest. In one embodiment, the surface of the object of interest is measured and virtually cut along the ultrasound plane.

### FIELD OF THE INVENTION

The present invention relates to laparoscopy image analysis and processing. In particular, the present invention relates to a calculation device for superimposing a laparoscopic image and an ultrasound image, a method of superimposing a laparoscopic image and an ultrasound image, a program element for superimposing a laparoscopic image and an ultrasound image, a computer-readable medium on which a program element is stored and a trocar comprising a depth-sensing imaging device.

### BACKGROUND OF THE INVENTION

The use of ultrasound in the operating room by surgeons is increasing, including the indications and use of ultrasound in laparoscopy and endoscopy. In abdominal laparoscopy, the abdominal wall is lifted from the internal organs by creating airtight incision and blowing in carbon dioxide at low pressure. A long, rigid rod-lens scope (the laparoscope) and light cord for illumination are then inserted to allow visual examination of the abdominal organs via displayed images that are shown on one or more monitor screens, allowing the operating staff to monitor the progress of the operation. Several trocars, hollow plastic tubes with an air-tight valve, called trocars, are placed in strategic locations to allow the easy insertion, removal and exchange of surgical laparoscopic instruments.

In current environments, ultrasound image data are presented on separate monitors. Positioning and orientating the laparoscopic ultrasound probe in a correct manner relative to the point of interest is of particular importance. Laparoscopic instruments are situated inside a trocar and move about a pivot point, which limits their spatial degrees of freedom and makes them awkward to manipulate. This difficulty is compounded for laparoscopic ultrasound by the fact that image data from the laparoscope and ultrasound images are displayed on separate monitors without indication of their spatial correlation. Correct positioning and orientation of the ultrasound probe therefore poses a challenging task even for experienced laparoscopists.

### SUMMARY OF THE INVENTION

There may be a need to provide for an improved displaying of laparoscopy images.

The object of the present invention is solved by the subject matter of the independent claims. Further embodiments and advantages of the invention are incorporated in the dependent claims.

The described embodiments similarly pertain to the calculation device for superimposing a laparoscopic image and an ultrasound image, the method of superimposing a laparoscopic image and an ultrasound image, the computer program element, the computer-readable medium and the trocar comprising a depth-sensing imaging device. Synergistic effects may arise from different combinations of the embodiments although they might not be described hereinafter in detail.

Technical terms are used by their common sense. If a specific meaning is conveyed to certain terms, definitions of terms will be given in the following in the context of which the terms are used.

According to a first aspect of the present invention, a calculation device for superimposing a laparoscopic image and an ultrasound image is presented. The calculation device is configured to receive a laparoscope image of a laparoscope and is configured to receive an ultrasound image of an ultrasound device, in particular of a laparoscopic ultrasound device. Furthermore, the calculation device is configured to receive a depth image of a depth-sensing imaging device, wherein the depth image comprises data defining a surface of an object of interest. The calculation device is configured to extract depth cue information or depth information from the received depth image. Moreover, the calculation device is configured to use the extracted depth cue information or depth information for superimposing the laparoscopic image and the ultrasound image to generate a superimposed image.

The calculation device of the present invention can extract depth cue information from the received depth image. In particular, depth cue information may thus be generated that involves knowledge of a surface of the relevant object, such as an organ surface within the field of view of the ultrasound and/or laparoscopy devices. Such depth cue information may be useful in obtaining an improved superimposed image.

For example, an overlay of the ultrasound image and the laparoscopic image in the superimposed image may be generated, which is more intuitive to a user. In other words, a superimposed image can be displayed to the user that is more intuitive since the image has or makes use of one or more depth cues derived from the position of a surface of an object of interest, such as an organ in the field of view. For example, data regarding the position of an organ surface may be used in generating depth cues in the form of certain visual elements to be visualized in the superimposed image, resulting in a superimposed image that is more intuitive to a user.

The calculation device may use the extracted depth cue information to adapt the ultrasound image and/or the laparoscopic image such that a superimposed image is generated which comprises one or more corresponding depth cues, like for example a shadow, and/or an occlusion/overlap.

In an embodiment the superimposed image has the perspective of the laparoscope image and the ultrasound image is overlaid onto the laparoscope image.

As will be explained hereinafter in more detail, different embodiments of depth cues, also in combination, may be used in the superimposed image which is generated by the calculation device. The use of a depth sensing imaging device and of the depth image thereof provides knowledge about the surface of an object of interest, e.g. of an organ, such that superimposing, i.e. overlaying, laparoscopic and ultrasound images or video streams can result in a very intuitive superimposed image, taking into account the location of the surface of one or more organs in the field of view.

In other words, the calculation device may make use of the knowledge about relative distances of the laparoscope, the laparoscopic ultra sound device and an object of interest to each other and to the depth-sensing device to improve the spatial perception of the user superimposed image. This knowledge can be extracted by the calculation device from the depth image.

Different depth cue information, i.e., depth cues, can be extracted by the calculation device from the depth image and can be used by the calculation device for or during the generation of the superimposed image. For example, a real shadow and/or a virtual shadow from a virtual light source can be calculated by the calculation device and can be used in the superimposed image to improve the user's perception. Alternatively or additionally, an occlusion, i.e., a realistic overlap of objects in the laparoscopic image, can be used as an exemplary embodiment of a depth cue in the context of the present invention. Based on the depth cue information extracted from the depth image it can be determined by the calculation device whether additional objects are in the scene and which object has a larger distance to the laparoscope. Hence the calculation device can calculate which object shall overlap which other objects to provide a realistic visual impression in the superimposed image. Alternatively or additionally, the calculation device may also generate a superimposed image with accommodation, e.g. simulated depth of field of the superimposed image with different sharpness for objects in different distances. Alternatively or additionally, convergence and binocular parallax are embodiments of depth cues that could be used when stereo cameras are applied in combination with stereo displays. Alternatively or additionally, movement parallax is another depth cue that could be used. When the laparoscope moves, the parallax changes. This movement parallax may also be used in the superimposed image in an embodiment of the present invention. In case 3-dimensional ultrasound is used and a relatively thick object is imaged, linear perspective may also be a depth cue that can be used by the calculation device.

In an embodiment, the ultrasound image is displayed in the superimposed image in a transparency mode which further enhances the 3D-perception of the user. The calculation device can be configured to calculate such a transparency mode of the ultrasound image.

Further, in the context of the present invention the term “image” shall comprise single, individual images but also continuous video streams. In particular, a laparoscopic video stream and an ultrasound video stream may be received by the calculation device for the extraction of depth cue information and the subsequent generation of a superimposed image. The depth cues come from the depth-sensing device which is in place in addition to the laparoscope and the ultrasound device. In the same way, the superimposed image may be an individual image or may be a plurality of images, e.g. a video stream consisting of a plurality of superimposed images.

Moreover, in the context of the present invention, the term “depth-sensing imaging device” may be seen as an intra-abdominal depth camera which is configured to measure, by means of imaging or scanning, the surface of one or more objects of interest, in particular an organ surface of an internal organ, during laparoscopy. In an example, the depth-sensing imaging device can further be configured to determine the position and orientation of the involved instruments, in particular of the laparoscope and the ultrasound device.

The skilled person is well aware of depth-sensing imaging devices. For example, the depth-sensing imaging device may comprise a structured light system including an infrared (IR) structured light projector, an IR camera, and a normal colour camera. For example, a system with Intel® RealSense technology may be used.

Thus, for instance, a projected IR light pattern is distorted in the IR image. From this distortion a distance between the camera and an organ surface can be calculated, which results in the depth image.

In another example, the depth-sensing imaging device may include a time-of-flight (TOF) camera, such as provided in a Microsoft® Kinect v2 system. Thus, for example, the time it takes for a light pulse to travel from the emitter to an organ surface and back to the image sensor is measured. From this measured time of flight it is also possible to create a depth image representing the organ surface.

A depth image generated by such a device is to be understood as an image that contains information relating to the distance of the surfaces of scene objects from a viewpoint.

The calculation device of the present invention may be part of a computer, like a desktop or laptop, or may be part of a larger calculation entity like a server. The calculation device may also be part of a medical imaging system. The calculation device may be connected with the depth-sensing imaging device which can be locatedin a trocar inserted into a patient, for example.

In accordance with the calculation device presented hereinbefore, a method of superimposing a laparoscopic image and an ultrasound image is presented. The method comprises the steps of providing a laparoscopic image of a laparoscope, providing an ultrasound image of an ultrasound device, providing a depth image of a depth-sensing imaging device, extracting depth cue information from the depth image and using the extracted depth cue information for superimposing the laparoscopic image and the ultrasound image to generate a superimposed image.

Further embodiments of the calculation device and the method will be presented hereinafter. The skilled person will understand that whenever an embodiment of the calculation device is explained in detail, a corresponding method is disclosed therewith as well.

According to an exemplary embodiment of the present invention, the calculation device is configured for determining a form and a location of a shadow in the superimposed image based on the extracted depth cue information. The calculation device is also configured for adapting the ultrasound image and/or the laparoscopic image such that the shadow is visualized in the superimposed image.

The shadow described in this embodiment may result from a real light source, like e.g. the light source positioned at the laparoscope, but may also result from a virtual light source. For example, in FIG. 7, an embodiment is shown in which an artificial shadow 701 is calculated by the calculation device and is displayed to the user in the superimposed image 700. In both cases, the position and extension of the light source as well as the position and orientation of the laparoscope and the position and orientation of the ultrasound device is provided to the calculation device. Based on the information contained in the depth image the calculation device can then calculate how the imaged scene looks like from the perspective of the laparoscope using the depth cues real and/or artificial shadows. The same holds true for other depth cues like e.g. overlaps/occlusions. These data, i.e., the mentioned position and orientation of the laparoscope and ultrasound device, may be extracted from the depth image of the depth-sensing device, but may also be provided by for example sensors at the laparoscope and/or at the ultrasound device. This may entail tracking the position and orientation of these devices with said sensors. The tracking data can then be provided to calculation unit of the present invention which processes these data for generating the superimposed image. In case the position and orientation data of the laparoscope and the ultrasound device shall be provided by the depth-sensing device, the field of view of this imaging device is sufficiently wide to include both the laparoscope and ultrasound instrument as depicted for example in FIG. 2.

According to another exemplary embodiment of the present invention, the calculation device is configured for determining a form and a location of an overlap/occlusion in the superimposed image based on the extracted depth cue information. The calculation device is further configured for adapting the ultrasound image and/or the laparoscopic image such that the overlap/occlusion is visualized in the superimposed image. Displaying such a realistic overlap/occlusion to the user in the superimposed image may also improve the 3-dimensional perception of the user when applying the calculated superimposed image as a navigation support during laparoscopy. Based on the distances of objects shown in the depth image to the depth-sensing device, the calculation device can calculate which object in the superimposed image has to overlap which other object in order to give the user a realistic impression of the overlay. Based on this information, the calculation device can then calculate how the respective depth cue must be shown in the superimposed image that is generated. An exemplary embodiment thereof is depicted in FIG. 10.

According to another exemplary embodiment, the ultrasound image visualizes a cross section of an object of interest in an ultrasound plane. Furthermore, the calculation device is configured for calculating a form and a position of a hole in the object of interest in the superimposed image. A corresponding adaption of the laparoscope image and/or the ultrasound image can be comprised as well. Displaying such a hole to the user in the superimposed image may also improve the 3-dimensional perception of the user. Such a hole may have different forms like for example the rectangular form described in the context of FIGS. 8 and 9. The hole which is shown in the superimposed image may extend from the surface of the object of interest into the inner part of the object of interest. This facilitates that the ultrasound image, which is overlaid over the laparoscopic image, is shown before a background which displays an inner part of the object of interest. Since this inner part of the object of interest is also depicted in the cross-sectional view that is provided by the ultrasound image, a superimposed image with depth cue is presented.

According to another exemplary embodiment of the present invention, the calculation device is configured for virtually cutting the object of interest along the ultrasound plane and for displaying the object of interest with the resulting cut in the superimposed image. Exemplary embodiments thereof will be described in the context of FIGS. 6 and 7. The resulting cut may show an outer surface of the object of interest as well as an inner part of the object of interest. One embodiment is thus to measure the surface of the object of interest, i.e. the surface of an organ, and virtually cut it along the ultrasound plane. This allows for the possibility to virtually stain the inner part of the object of interest with a color that is different from the color of the outer surface of the object of interest. This may further improve the 3-dimensional perception of the user when using the superimposed image.

According to another exemplary embodiment of the present invention, the calculation device is configured for receiving data about a position and an extension of a virtual light source. For example, this data may be provided by a user to the calculation device. The calculation device is further configured for determining a form and a location of a virtual shadow in the superimposed image based on the extracted depth cue information and based on the position and the extension of the virtual light source. The calculation device is configured for adapting the ultrasound image and/or the laparoscopic image such that the artificial shadow is visualized in the superimposed image. This embodiment may particularly be combined with the embodiment explained herein before in which the object of interest is virtually cut along the ultrasound plane. Calculating and displaying such an artificial shadow in the area of the cut, e.g. artificial shadow 701 shown in FIG. 7, may further improve the 3-dimensional perception of the user.

According to another exemplary embodiment of the present invention, the calculation device is configured for extracting the spatial position and the orientation of the laparoscope and the spatial position and the orientation of the ultrasound device from the depth image. Moreover, the calculation device is configured for transforming the extracted spatial position and the extracted orientation of the laparoscope and the extracted spatial position and the extracted orientation of the ultrasound device into a common coordinate system. The main principles of registering coordinate systems are generally known to the skilled person. The calculation device of the present invention may particularly be configured to calculate such registrations as known from the prior art, e.g. from IGSTK Image-Guided Surgery Toolkit—An open Souce C++ Software Library, edited by Kevin Cleary, Patrick Cheng, Andinet Enquobahrie, Ziv Yaniv, Insight Software Consortium 2009, or from J. Yanof, C. Bauer, S. Renisch, J. Kriicker, J. Sabczynski, Image-Guided Therapy (IGT): New CT and Hybrid Imaging Technologies, in Advances in Healthcare Technology, edited by G. Spekowius, T. Wendler, Springer, 2006.

According to another exemplary embodiment of the present invention, the calculation device is configured for receiving data about a position of a head-mountable augmented-reality device. The calculation device is further configured for co-registering the position of the head-mountable augmented-reality device with the common coordinate system. The calculation device is also configured for transmitting the superimposed image to the head-mountable augmented-reality device. Therefore, the superimposed image can also be displayed on a head-mounted reality device worn by the operating staff if the position of these devices is also captured and co-registered with the common coordinate system as mentioned herein before. A further option is to display the superimposed image on a device such as a tablet computer positioned in the user's field of view. In the latter case, both the user's eye positions and direction of gaze as well as the location and orientation of the display device are provided by a respective device or by the user and are co-registered with the before described common coordinate system by the calculation unit.

According to another aspect of the present invention, a program element for superimposing a laparoscopic image and an ultrasound image is presented.

The computer program element may be part of a computer program, but it can also be an entire program by itself. For example, the computer program element may be used to update an already existing computer program to get to this aspect of the present invention.

According to another aspect of the present invention, a computer-readable medium on which a computer program element for superimposing a laparoscopic image and an ultrasound image is stored is presented.

The computer-readable medium may be seen as a storage medium, such as for example a USB stick, a CD, a DVD, a data storage device, a hard disk, or any other medium on which a computer program element as described above can be stored.

According to another aspect of the present invention, a trocar comprising a depth-sensing imaging device is presented. The depth-sensing imaging device may be attached to the exterior surface of the trocar which are typically inserted into the intra-abdominal working space. In another embodiment the trocar comprises the depth-sensing imaging device inside its housing. In an embodiment the trocar together with the calculation device of the present invention may be combined in a system. The aspect of the present invention relating to the trocar comprising the depth-sensing imaging device can explicitly combined with each other embodiment of the present invention mentioned herein. The depth-sensing imaging device of the trocar may be connected, wire-bound or wireless, with a calculation device of the present invention. The calcualtion device may then carry out the method of the present invention as described herein.

It may be seen as an aspect of the present invention to use depth information gathered from a depth image of a laparoscopic depth-sensing imaging device to generate a superimposed image comprised of an ultrasound image and a laparoscopic image. This may enhance the 3-dimensional perception of the superimposed image shown to the user. Since the ultrasound shows information from within the object of interest, while the laparoscope shows the surface of the object of interest, a naïve overlay of the ultrasound image over the laparoscope image, as done in the prior art, may look unnatural, since no depth cues are taken into account. In contrast thereto the present invention allows displaying the ultrasound image correctly aligned in space with respect to the laparoscopic image with correct depth cues.

These and other features of the invention will become apparent from and elucidated with reference to the embodiments described hereinafter.

### BRIEF DESCRIPTION OF THE DRAWINGS

Exemplary embodiments of the invention will be described in the following drawings. Identical reference numerals are used for similar or identical elements shown in the following figures.

FIG. 1 schematically shows a flow diagram of a method of superimposing a laparoscopic image and an ultrasound image according to an aspect of the present invention.

FIG. 2 schematically shows a set up with calculation device for superimposing a laparoscopic image and an ultrasound image together with a laparoscope, an ultrasound device and a depth-sensing device.

FIG. 3 schematically shows a real view from a laparoscope.

FIG. 4 schematically shows a superimposed image of a laparoscopic image and an ultrasound image with position correct overlay without transparency mode.

FIG. 5 schematically shows a superimposed image of a laparoscopic image with an ultrasound image with a transparent overlay, transparency mode, and a correct position of the ultrasound image.

FIG. 6 schematically shows a superimposed image with a virtual cut plane.

FIG. 7 schematically shows a superimposed image with a virtual cut plane and with an artificial shadow.

FIG. 8 schematically shows a superimposed image with a hole with no transparency mode and no artificial shadow.

FIG. 9 schematically shows a superimposed image with a hole with transparency mode and with an artificial shadow.

FIG. 10 schematically shows a superimposed image with a grasper as an additional object in the scene and an overlay between the grasper and the ultrasound image as depth cue information.

### DETAILED DESCRIPTION OF EMBODIMENTS

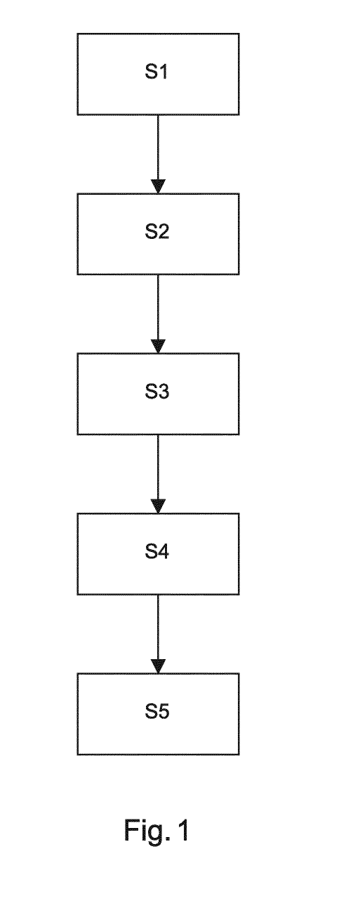

FIG. 1 schematically shows a method of superimposing a laparoscopic image and an ultrasound image according to an aspect of the present invention. In a first step S1, the laparoscopic image of a laparoscope is provided. Providing an ultrasound image of an ultrasound device is presented in step S2. A depth image of a depth-sensing device is provided in step S3. Extracting depth cue information from the provided depth image is shown in FIG. 1 by step S4. The extracted depth cue information is used for superimposing the laparoscopic image and the ultrasound image to generate the superimposed image in step S5. This method can be carried out by a calculation unit as presented hereinbefore and hereinafter. Several different method steps may be added to this method of FIG. 1 according to several other method embodiments of the present invention. For example, determining a form and a location of a shadow and/or of an occlusion, as described hereinbefore, can be part of a method embodiment. Also the step of adapting the ultrasound image and/or the laparoscopic image are possible further method steps. In another method embodiment, virtually cutting the object of interest along the ultrasound plane and displaying the object of interest with the resulting cut in the superimposed image is a further method step. In another embodiment, the step of virtually staining the inner part of the object of interest with a color that is different from the color of the outer surface of the object of interest is an additional method step.

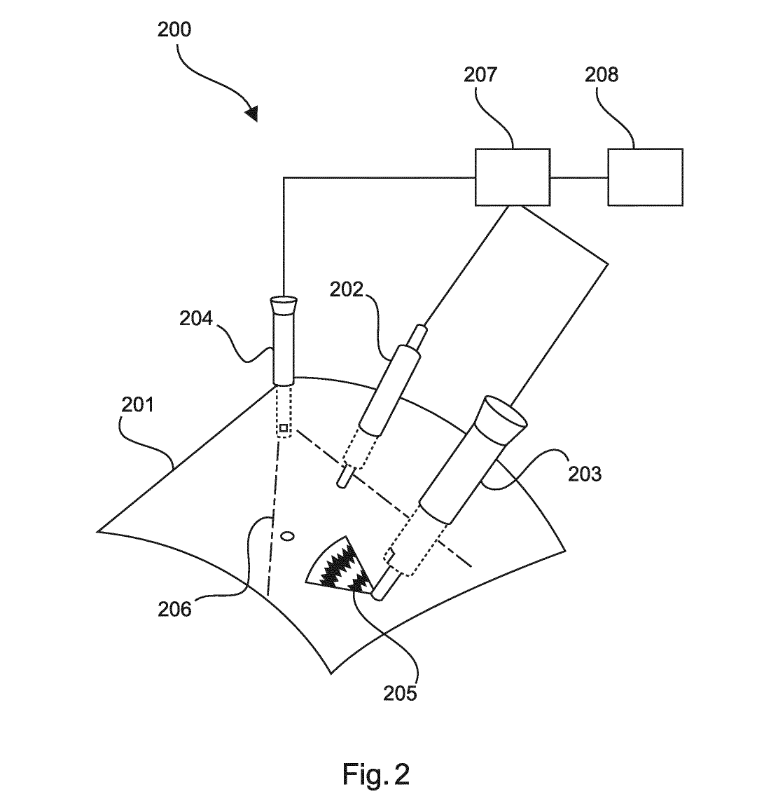

The method embodiments described before may be combined with the steps of extracting the spatial position and the orientation of the laparoscope and the ultrasound device from the depth image. Moreover, transforming the extracted spatial position and the extracted orientation of the laparoscope and the extracted spatial position and the extracted orientation of the ultrasound device into a common coordinate system by the calculation unit may be part of a supplemented embodiment of the method of FIG. 1. In particular, such extraction and transformation can be done by the calculation device by processing the real time image feed of the depth-sensing device. The method of FIG. 1 may be carried out when using the calculation device described hereinafter in the context of FIG. 2.

FIG. 2 schematically shows a setup 200 in which a calculation device 207 according to an exemplary embodiment of the present invention is used. FIG. 2 shows the abdominal surface 201 as well as the laparoscope 202, the ultrasound imaging device 203 and the depth-sensing device 204. The ultrasound image 205 generated by the ultrasound device 203 is shown in FIG. 2 as well. The angle of view 206 of the depth-sensing device 204 in this embodiment is wide enough to include both the laparoscope and the ultrasound instrument. Therefore, the depth image generated by the device 204 comprises data about the spatial position and orientation of the laparoscope 202 and of the ultrasound device 203. The calculation device 207 may thus be configured for extracting the spatial position of each device and the orientation of each device and may further be configured for transforming the extracted positions and extracted orientations into a common coordinate system. The calculation device 207 may also be configured to transmit the superimposed image to the display 208. In this and each other embodiment of the present invention, the calculation device 207 may be provided with information how the perspective of the laparoscopic image is relative to the perspective of the ultrasound image and relative to the perspective of the depth image. This information can be extracted from for example the depth image but also other means like sensors which track the position and orientation of the laparoscope, the ultrasound device and/or the depth-sensing device may be used.

The calculation device 207 can be configured for warping the ultrasound image to fit a focal length of the laparoscope and image distortions. The technical effect resulting therefrom is a correction of optical abrasions or optical errors caused by optical elements used in the laparoscope.

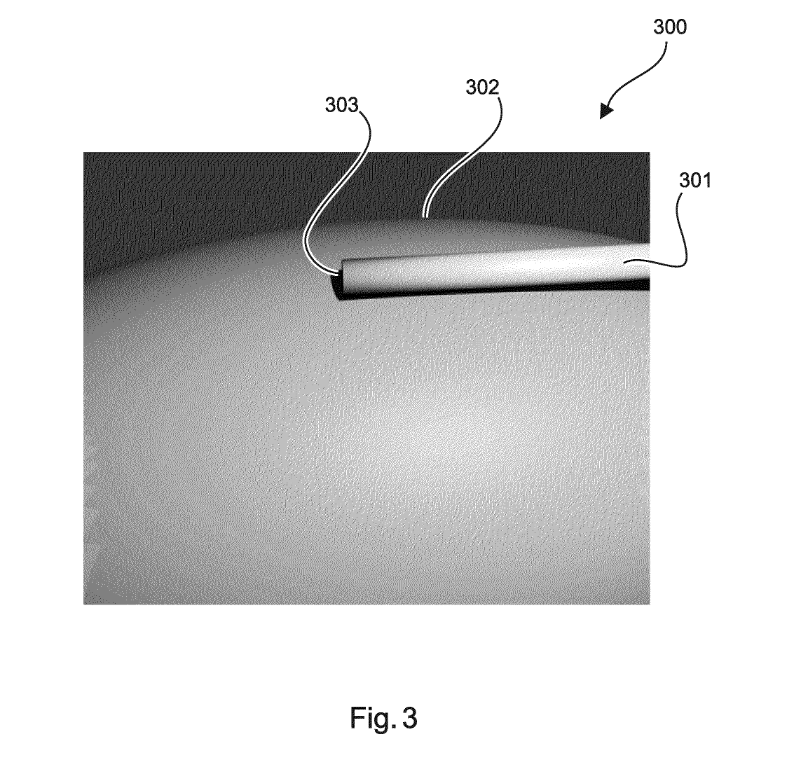

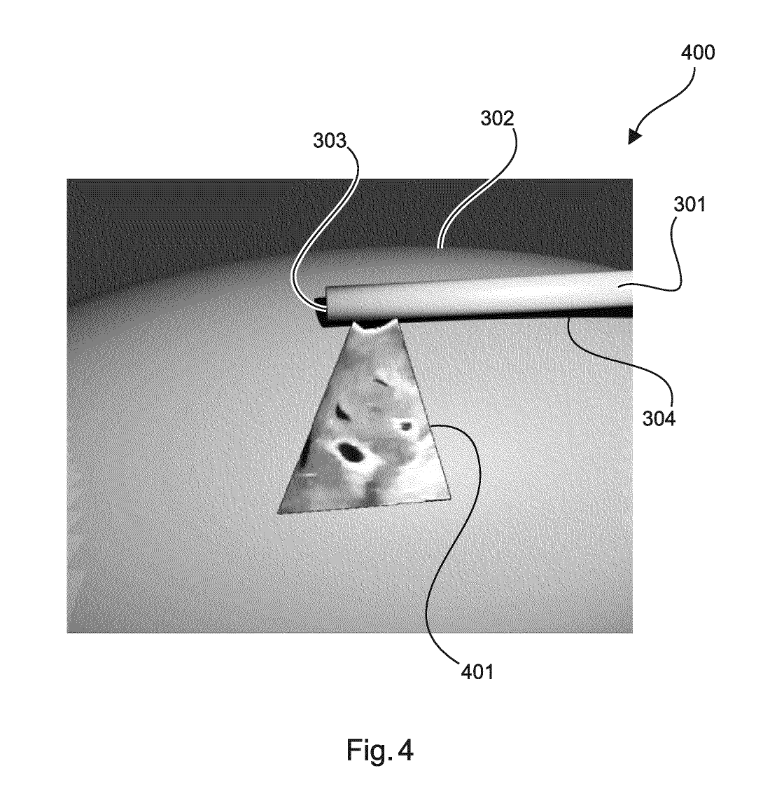

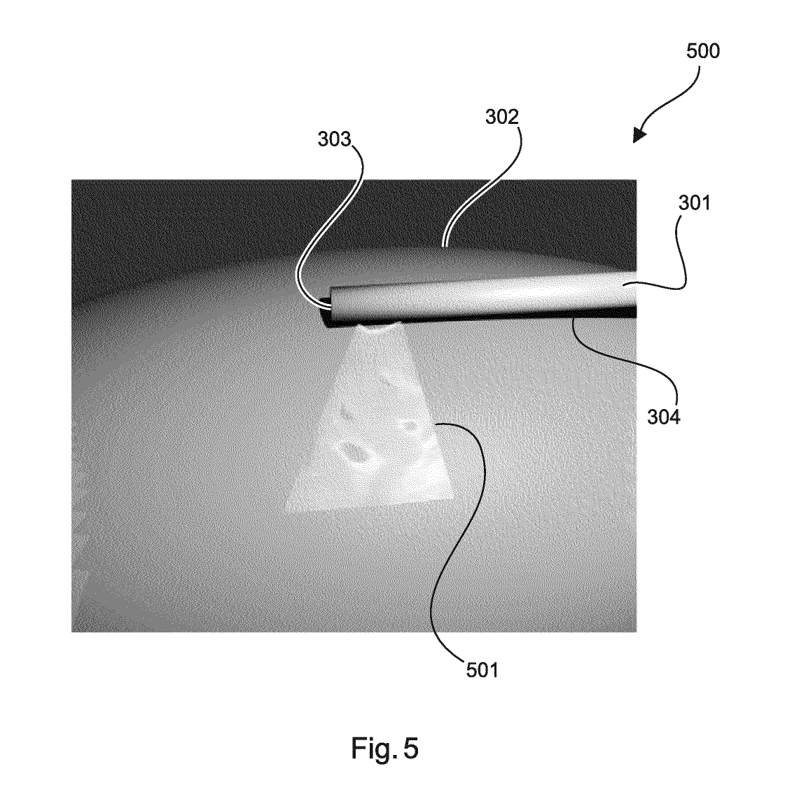

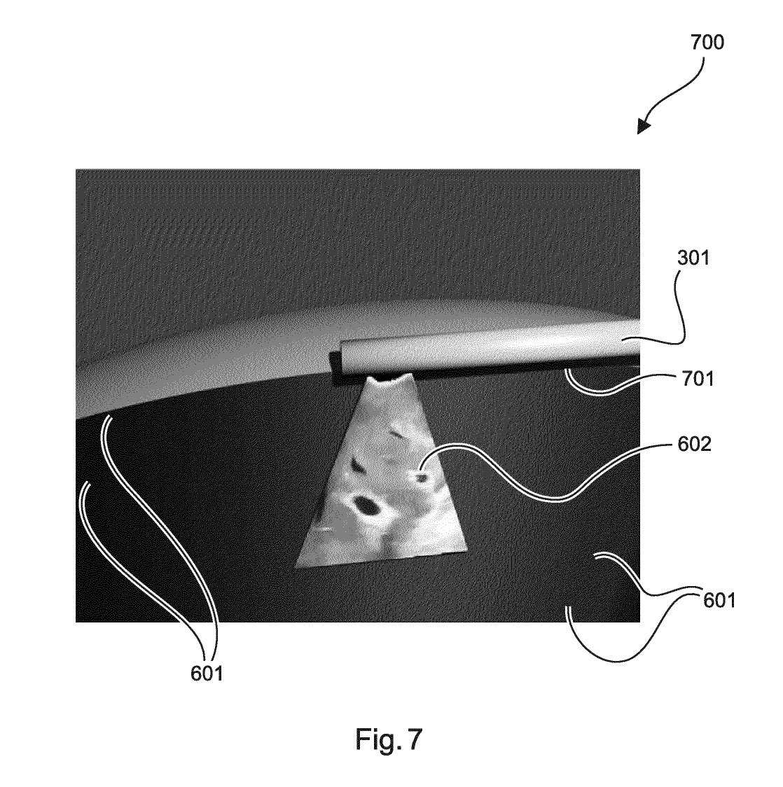

FIG. 3 schematically shows a real image 300 of a laparoscope in which a laparoscopic ultrasound device 301 is depicted over the surface 302 of an object of interest, e.g. of an organ. Since a light source is attached to the laparoscope, a shadow 303 is comprised as well. In addition to FIG. 3, FIG. 4 schematically shows a superimposed image 400 in the perspective of the laparoscope with a superimposed ultrasound image 401. This superimposed image 400 may be generated by the calculation device according to the present invention. The ultrasound image 401 is shown at the correct position with respect to the surface 302 of the object of interest and with respect to the ultrasound device 301 since data from the depth image of the depth-sensing device are used to generate this overlay by the calculation device of this embodiment. After a calibration of the laparoscope camera its camera parameters are known. This allows calculating the projection of objects with known shape into the image of the laparoscope. After a calibration of the ultrasound it is known for each pixel of the ultrasound image from which position in space relative to the ultrasound scan head it comes from. Therefore, it is possible to calculate the projection of a pixel of the ultrasound image into the laparoscopes image. This allows the position correct overlay. Additionally, the calculation device of the present invention can then calculate different depth cues as described herein, e.g. the depth cues used in the embodiments of FIGS. 5 to 10, and amend the image of FIG. 4 accordingly.

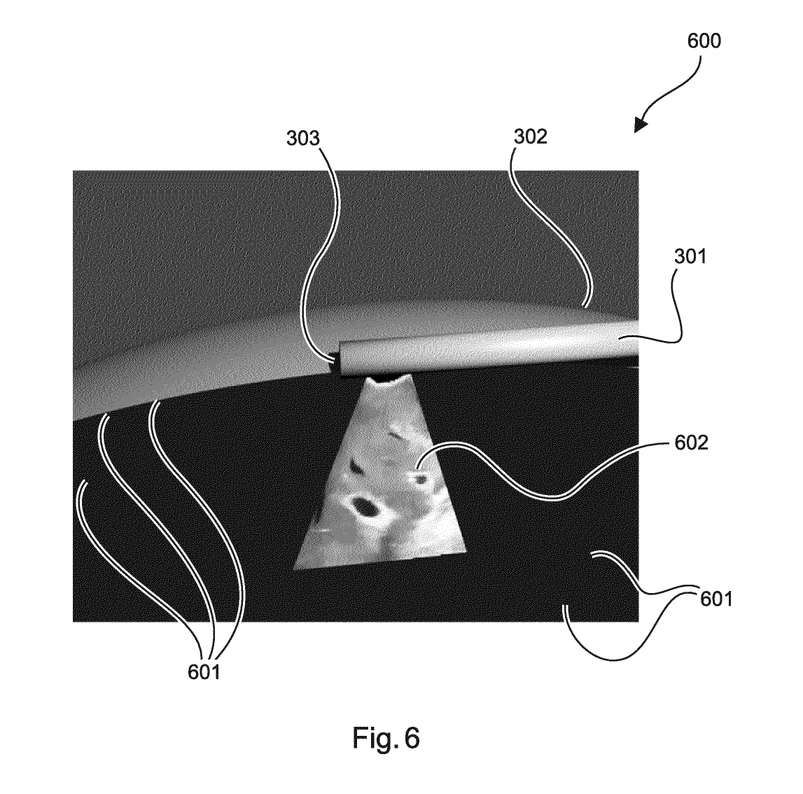

FIG. 5 shows a superimposed image 500 calculated by a calculation device according to an embodiment of the present invention. In this embodiment, the ultrasound image 501 is provided in transparency mode, i.e. as a transparent overlay over the laparoscopic image thereby enhancing the depth effect. This may further increase the 3-dimensional perception of the user when using this superimposed image. Thus, it might be understood that for the transparency mode the calculation device of the present invention may adjust the original opaque US image data (see FIG. 4) to be more or less transparent with a maximum at fully transparency, i.e. an invisible US image. Additionally, depth cues may be added to the image of FIG. 5 as has been described herein before and hereinafter. According to another exemplary embodiment, FIG. 6 shows a superimposed image 600 generated by an embodiment of the calculation device of the present invention. The superimposed image 600 of FIG. 6 shows a virtual cut plane calculated by the calculation device. The cut extends from the surface 302 of the object of interest as determined by means of the depth-sensing imaging device, and is visualized by the dark surface 601.

The calculation device of the corresponding embodiment of the present invention is thus configured for virtually cutting the object of interest along the ultrasound plane of the us image 602. The border between the object surface 302 and the virtual cut plane is determined using the depth image from the depth-sensing imaging device.

Furthermore, the calculation device is configured for virtually staining the inner part of the object of interest with a color that is different from the color of the outer surface of the object of interest. As can be gathered from FIG. 6, the surface 302 is shown with a different color as compared to the inner part of the object of interest that is graphically represented by the dark surface 601. By providing this virtual cut and the staining of the inner part of the object of interest, the ultrasound image 602 is overlaid in a more intuitive way over the laparoscopic image.

In a further exemplary embodiment, a superimposed image 700 is generated by a calculation device according to the present invention. In this embodiment, the superimposed image 700 in addition to the embodiment of FIG. 6 comprises an artificial shadow 701 in the area where the cut is located. The calculation unit of the embodiment of the present invention which generates the superimposed image is thus configured for determining the form and location of the artificial shadow 701 in the superimposed image 700 based on the extracted depth cue information and based on the position and the extension of an artificial light source. The position and the extension of the artificial light source may be provided by the user. The calculation device then adapts the ultrasound image and/or the laparoscopic image such that the artificial shadow 701 is visualized in the superimposed image 700.

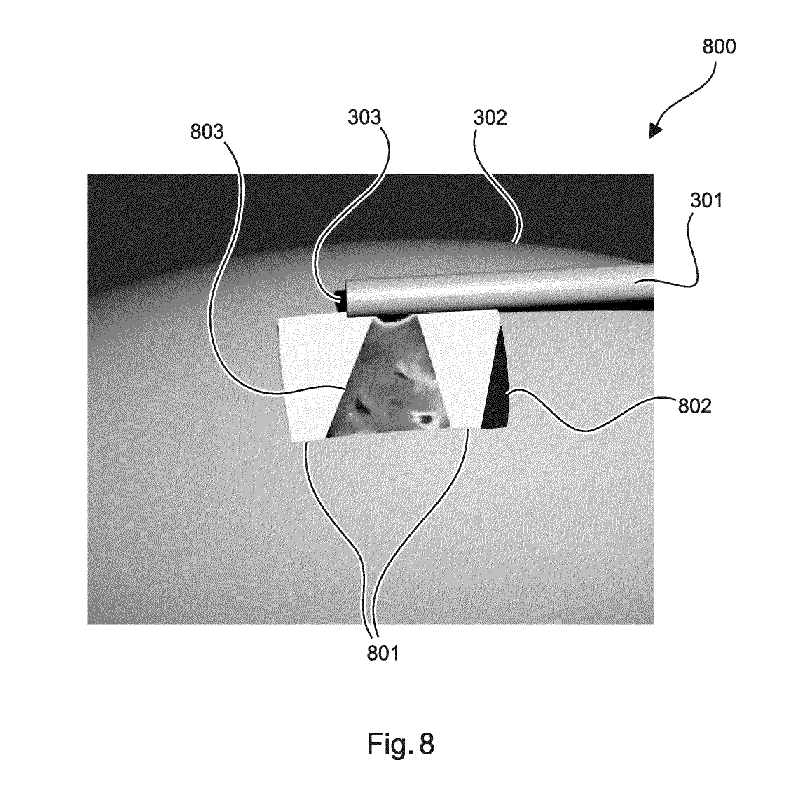

FIG. 8 shows another superimposed image 800 generated by a calculation device of an embodiment of the present invention. Superimposed image 800 shows a hole 801 in the object of interest. The calculation device of this embodiment of the present invention has calculated the form and the position of the hole 801. The ultrasound image 803 overlaps the hole 801. The superimposed image 800 does not show the ultrasound image 803 in transparency mode and does not comprise artificial shadows.

The “circumference” of the hole is calculated by the calculation device calculating the intersection of a “block”, which is attached to the ultrasound, with the surface of the organ of interest as measured by the depth-sensing camera. Each side of the block may be colored differently in order to provide a realistic shadow effect inside the hole. Within the circumference of the hole, all pixels of the original laparoscope image are deleted by the calculation device.

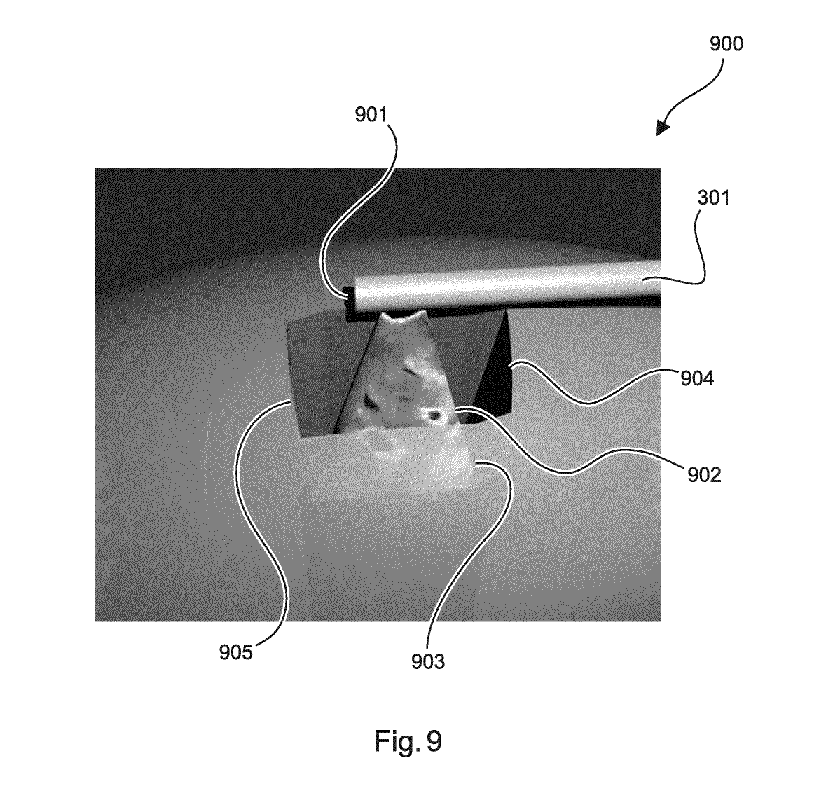

FIG. 9 schematically shows a further superimposed image 900 generated by a calculation device according to an exemplary embodiment of the present invention. In the superimposed image 900, the ultrasound image 902 shows an artificial shadow 901 below the ultrasound device 301 and at the right side 904 of the hole 905. The ultrasound image 902 is also provided in a transparency mode such that a lower part 903 of the ultrasound image can still be seen in the superimposed image 900. This is very similar to FIG. 8. Inside the circumference of the hole, all pixels of the original laparoscope image are completely transparent/deleted by the calculation device, while outside of the circumference of the hole the laparoscope image is made transparent, thus showing the walls of the hole.

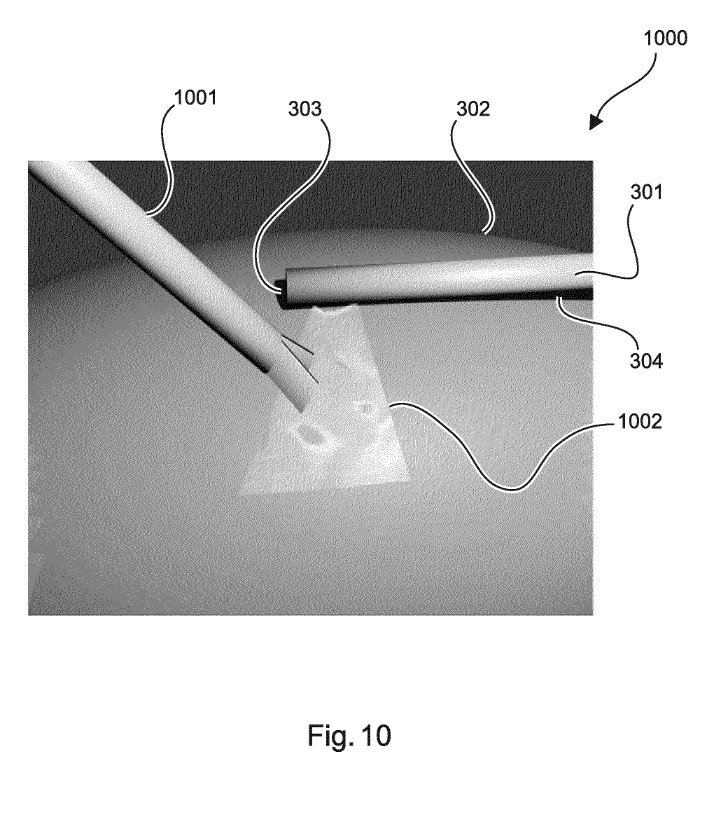

FIG. 10 schematically shows a superimposed image 1000 in which a grasper 1001 is shown as an additional object. Based on the depth information extracted from the depth image, the calculation device of an embodiment of the present invention calculates that grasper 1001 has a shorter distance to the laparoscope as compared to the position of the ultrasound image 1002. Therefore, grasper 1001 overlaps the ultrasound image 1002 such that a realistic intuitive superimposed image 1000 can be presented to the user.

1. A calculation device for superimposing a laparoscopic image and an ultrasound image,
wherein the calculation device is configured to receive a laparoscope image of a laparoscope,
wherein the calculation device is configured to receive an ultrasound image of an ultrasound device,
wherein the calculation device is configured to receive a depth image of a depth-sensing imaging device, wherein the depth image comprises data defining a surface of an object of interest,
wherein the calculation device is configured to extract depth cue information from the depth image, and
wherein the calculation device is configured to use the extracted depth cue information for superimposing the laparoscopic image and the ultrasound image to generate a superimposed image.

2. Calculation device according to claim 1,
wherein the calculation device is configured to determine a form and a location of a shadow to be visualized in the superimposed image based on the extracted depth cue information.

3. Calculation device according to claim 1,
wherein the calculation device is configured to determine a form and a location of an occlusion to be visualized in the superimposed image based on the extracted depth cue information.

4. Calculation device according to claim 1,
wherein the ultrasound image visualizes a cross section of the object of interest in an ultrasound plane,
wherein the calculation device is configured to calculate a form and a position of a hole in the object of interest to be visualized in the superimposed image.

5. Calculation device according to claim 1,
wherein the ultrasound image visualizes a cross section of the object of interest in an ultrasound plane, and
wherein the calculation device is configured for virtually cutting the object of interest along the ultrasound plane and for displaying the object of interest with the resulting cut in the superimposed image.

6. Calculation device according to claim 5,
wherein superimposed image with the resulting cut shows an outer surface of the object of interest and an inner part of the object of interest, and
wherein the calculation device is configured to virtually stain the inner part of the object of interest with a color that is different from the color of the outer surface of the object of interest.

7. Calculation device according to claim 6,
wherein the calculation device is configured to receive data about a position and an extension of an virtual light source,
wherein the calculation device is configured to determine a form and a location of an artificial shadow in the superimposed image based on the extracted depth cue information and based on the position and the extension of the virtual light source, and
wherein the calculation device is configured to adapt the ultrasound image and/or the laparoscopic image such that the artificial shadow is visualized in the superimposed image.

8. Calculation device according to claim 1,
wherein the depth image comprises data about a spatial position and/or an orientation of the laparoscope, and
wherein the depth image comprises data about a spatial position and/or an orientation of the ultrasound device.

9. Calculation device according to claim 8,
wherein the calculation device is configured to extract the spatial position and the orientation of the laparoscope and the spatial position and the orientation of the ultrasound device from the depth image data, and
wherein the calculation device is configured to transform the extracted spatial position and the extracted orientation of the laparoscope and the extracted spatial position and the extracted orientation of the ultrasound device into a common coordinate system.

10. Calculation device according to claim 9,
wherein the calculation device is configured to receive data about a position of a head-mountable augmented-reality device,
wherein the calculation device is configured to co-register the position of the head-mountable augmented-reality device with the common coordinate system, and
wherein the calculation device is configured to transmit the superimposed image to the head-mountable augmented-reality device.

11. Calculation device according to claim 9,
wherein the calculation device is configured to receive data about a user's eye position and direction of gaze;
wherein the calculation device is configured to receive a spatial position and an orientation of a computer positioned display device, and
wherein the calculation device is configured to co-register the captured user's eye position and the captured direction of gaze and the captured spatial position and orientation of the computer positioned display device with the common coordinate system, and
wherein the calculation device is configured to transmit the superimposed image to the computer positioned display device.

12. Calculation device according to claim 11,
wherein the calculation device is configured to warp the ultrasound image to fit a focal length of the laparoscope and image distortions.

13. Method of superimposing a laparoscopic image and an ultrasound image, the method comprising the steps,
providing a laparoscopic image of a laparoscope,
providing an ultrasound image of an ultrasound device,
providing a depth image of a depth-sensing imaging device, wherein the depth image comprises data defining a surface of an object of interest,
extracting depth cue information from the depth image, and
using the extracted depth cue information for superimposing the laparoscopic image and the ultrasound image to generate a superimposed image.

14. A computer program product comprising a set of instructions for causing a calculation device for superimposing a laparoscopic image and an ultrasound image to carry out a method according to claim 13.

15. A computer-readable medium having stored thereon a computer program product according to claim 14.
